(19) United States
(12) Patent Application Publication
(10) Pub. No.: US 2002/0028942 A1
(43) Pub. Date: Mar. 7, 2002

(54) Novel process and compound

(76) Inventors: Victor Witold Jacewicz, Tonbridge (GB); Neal Ward, Tonbridge (GB)

Correspondence Address: GLAXOSMITHKLINE, Corporate Intellectual Property - UW2220, P.O. Box 1539, King of Prussia, PA, 19406-0939, US

(21) Appl. No.: 09/924,989

(22) Filed: Aug. 8, 2001

Related U.S. Application Data
(63) Continuation of application No. 09/365,938, filed on Aug. 2, 1999.

(30) Foreign Application Priority Data
Jan. 15, 1997 (GB) 9700693.6

(51) Int. Cl. C 07D 4 7/02; A61K 31/445; A61K 31/4525

(52) U.S. Cl. 546/197; 514/321

(57) ABSTRACT

Paroxetine hydrochloride is obtained in an easily soluble form suitable for preparing stable aqueous solutions, suitable for parenteral use, by freeze-drying solutions prepared by dissolving paroxetine free base in aqueous hydrochloric acid, or of paroxetine hydrochloride hemihydrate or other hydrate/solvate/amorphous forms of paroxetine hydrochloride.

The present invention relates to a process for the preparation of a pharmaceutically active compound, and to the use of the so-prepared compound in therapy. In particular this invention is concerned with the preparation of an easily-soluble form of paroxetine hydrochloride.

Pharmaceutical products with antidepressant and anti-Parkinson properties are described in U.S. Pat. No. 3,912,743 and U.S. Pat. No. 4,007,196. An especially important compound among those disclosed is paroxetine, the (−)trans isomer of 4-(4′-fluorophenyl)-3-(3′,4′-methylenedioxyphenoxymethyl)-piperidine. This compound is used in therapy as the hydrochloride salt for the treatment and prophylaxis of inter alia depression, obsessive compulsive disorder (OCD) and panic.

Paroxetine hydrochloride has been described in the literature as a crystalline hemihydrate (see EP-A-0223403 of Beecham Group) and as various crystalline anhydrate forms (see WO96/24595 of SmithKline Beecham plc). These known forms are not ideally suited for all pharmaceutical applications. For example parenteral formulations are advantageously prepared by sterile filtration of a liquid feedstock, and the low solubility in water of paroxetine hydrochloride, particularly in the hemihydrate form, is well documented. Furthermore, liquids can be dispensed into unit dosages more conveniently and precisely than solids, and the use of liquid feedstocks reduces hazards associated with dust. In addition, the known solid forms of paroxetine hydrochloride are relatively insoluble and are slow to dissolve completely.

There remains a need for a form of paroxetine hydrochloride with desirable properties for specific applications such as, for example, parenteral dosage.

According to a first aspect of the invention, there is provided a process for preparing an easily soluble form of paroxetine hydrochloride which comprises freeze-drying a solution of paroxetine hydrochloride.

The feedstock for freeze-drying may be prepared conveniently by, for example, dissolution of paroxetine free base in aqueous hydrochloric acid, although other solid forms of paroxetine hydrochloride may also be dissolved. For example, the feedstock may be prepared by dissolving amorphous paroxetine hydrochloride or a crystalline paroxetine hydrochloride anhydrate, hydrate or solvate in suitable solvent. The solvent used may be pure water or a mixture of water with compatible organic solvents. Some heating may be used to achieve and maintain complete solution, though once dissolved and in the absence of seeds of a crystalline form, aqueous solutions are stable at ambient temperature for many days. Suitable concentrations of paroxetine hydrochloride for freeze-drying are in the range 1 to 30% by weight, preferably in the range 5% to 20% by weight.

Surprisingly it has been found that aqueous solutions of normally sparingly soluble paroxetine hydrochloride hemihydrate that are of a suitable concentration for freeze-drying can be prepared, and that they are sufficiently stable for sterile filtration and liquid processing, even though they are highly supersaturated with respect to paroxetine hydrochloride hemihydrate. Processing at elevated temperature may be employed to reduce the risk of unwanted crystallisation during processing but is not essential.

Freeze-drying paroxetine hydrochloride has an advantage over existing preferred manufacturing procedures, such as crystallisation, in that pure water may be used as a solvent, thus obviating the need for expensive and environmentally undesirable organic solvents. Not withstanding this advantageous procedure, this invention includes the preparation of freeze-dried paroxetine hydrochlorides from solvent mixtures of water and organic solvents conventionally used in freeze-drying processes. If solvents are used, they are removed efficiently during freeze-drying, as are residual solvents from earlier process steps.

The solution is preferably freeze-dried in vials, so as to be in a sterile environment ready for dissolution. Alternatively, other conventional techniques such as drum or tray freeze-drying may be used.

The product obtained by the above freeze-drying process is a stable crisp solid suitable for pharmaceutical applications.

Accordingly, in a second aspect, this invention provides freeze-dried paroxetine hydrochloride.

The product is homogenous without any further processing and is obtained in 100% yield, a higher yield than is possible in any crystallisation process. The freeze-dried paroxetine hydrochloride product dissolves quickly and completely, and is particularly suited for parenteral use since low temperatures are used for isolation and thermal degradation processes are avoided.

It has been found that aqueous solutions of suitable concentration for parenteral dosage can be obtained by dispersing freeze-dried paroxetine hydrochloride in water, and that these reconstituted solutions are sufficiently stable for liquid dispensing even though they are supersaturated with respect to paroxetine hydrochloride hemihydrate.

The freeze-dried product of this invention may be formulated for therapy in the dosage forms described in EP-A-0223403 or WO96/24595. However the easily soluble nature of freeze dried paroxetine hydrochloride makes it especially suitable for the preparation of solutions for parenteral use, and the reconstituted sterile solution may be made up to approx. 2% solution for formulation in parenteral unit dosage form.

Therapeutic uses of the paroxetine product of this invention include treatment of: alcoholism, anxiety, depression, obsessive compulsive disorder, panic disorder, chronic pain, obesity, senile dementia, migraine, bulimia, anorexia, social phobia, pre-menstrual syndrome (PMS), adolescent depression, trichotillomania, dysthymia, and substance abuse, referred to below as “the disorders”.

Accordingly, the present invention also provides:

a pharmaceutical composition for treatment or prophylaxis of the disorders comprising freeze-dried paroxetine hydrochloride and a pharmaceutically acceptable carrier or an aqueous solution of reconstituted freeze-dried paroxetine hydrochloride;

the use of freeze-dried paroxetine hydrochloride to manufacture a medicament in solid or reconstituted liquid form for the treatment or prophylaxis of the disorders; and

a method of treating the disorders which comprises administering an effective or prophylactic amount of freeze-dried paroxetine hydrochloride as a solid oral composition or as a reconstituted aqueous oral or parenteral composition to a person suffering from one or more of the disorders.

The invention is illustrated by the following Example.

### EXAMPLE

Paroxetine hydrochloride hemihydrate (10 g) was dissolved in hot water (125 ml) and the cooled solution freeze dried in an Edwards Supermodulo model 12K freeze drier, using a vacuum of 5×10⁻¹ torr, with the freezing chamber set at −55° C. The resulting fine white powder was shown by NMR to be substantially pure paroxetine hydrochloride. The X-ray powder diffractogram consisted of a single very broad band centred at about 20° 2 theta, confirming the non-crystalline nature of the product. The water content was about 1%.

What is claimed is:

1. A process for preparing an easily soluble form of paroxetine hydrochloride which comprises freeze drying a solution of paroxetine hydrochloride.

2. A process according to claim 1, in which the feedstock for freeze drying is prepared by dissolution of paroxetine free base in aqueous hydrochloric acid.

3. A process according to claim 1, in which the feedstock is prepared by dissolving amorphous paroxetine hydrochloride or a crystalline paroxetine hydrochloride anhydrate, hydrate or solvate in a suitable solvent.

4. A process according to claim 1 in which the solvent is pure water or a mixture of water with compatible organic solvents.

5. Freeze-dried paroxetine hydrochloride.

6. A pharmaceutical composition for treatment or prophylaxis of the disorders comprising freeze-dried paroxetine hydrochloride and a pharmaceutically acceptable carrier or an aqueous solution of reconstituted freeze-dried paroxetine hydrochloride.

7. A method of treating the disorders which comprises administering an effective or prophylactic amount of freeze-dried paroxetine hydrochloride as a solid oral composition or as a reconstituted aqueous oral or parenteral composition to a person suffering from one or more of the disorders.
